(19) United States
(12) Patent Application Publication
(10) Pub. No.: US 2006/0089658 A1
(43) Pub. Date: Apr. 27, 2006

(54) Method and apparatus for treating abnormal uterine bleeding

(76) Inventor: Douglas C. Harrington, San Jose, CA (US)

Correspondence Address: CROCKETT & CROCKETT, 24012 CALLE DE LA PLATA, SUITE 400, LAGUNA HILLS, CA, 92653, US

(21) Appl. No.: 10/971,299

(22) Filed: Oct. 21, 2004

(51) Int. Cl. A61B 17/42

(52) U.S. Cl. 606/119; 606/193

(57) ABSTRACT

A method and device for creating amenorrhea in women. A porous biomaterial implant is positioned into the ablated uterine cavity of a female patient. Once positioned in the uterine cavity, the porous biomaterial implant expands into its preformed shape or is inflated within the uterine cavity. The ablated uterine tissue then grows within the porous implant. Prior to insertion of the implant, the uterine cavity is prepared by performing endometrial ablation to at least the inferior or lower portion of the uterine cavity. Placement of the porous biomaterial causes the uterine cavity walls to coapt, achieving complete occlusion of the uterine cavity and prevention of endometrial regrowth.

### FIELD OF THE INVENTIONS

The inventions described below relate to an apparatus and method for treating Abnormal Uterine Bleeding (AUB). In particular, the invention is directed to placement of a porous biomaterial implant introduced into the pre-treated uterine cavity of a female patient to reduce or eliminate the symptoms of AUB or serve as a method of contraception.

### BACKGROUND OF THE INVENTIONS

Abnormal Uterine Bleeding (AUB), also known as menorrhagia or dysfunctional uterine bleeding (DUB) is a condition characterized by excessive and prolonged menstrual bleeding. This condition can lead to extreme discomfort and embarrassment that can severely affect a woman's overall quality of life. This condition affects approximately one out of every five women between the ages of 35 and 50. Clinically, menorrhagia can be defined as a menstrual period that lasts for more than seven days or which produces blood loss in excess of 80 milliliters (mL) per menstrual cycle, where normal menstruation produces approximately 35 to 50 mL of blood loss.

AUB is caused by hormonal changes, or by a variety of different medical problems such as uterine fibroids, pelvic inflammatory disease, uterine hyperplasia and uterine cancer. The most common cause of AUB resides in the endometrium, which is the inner lining of the uterus. Women who suffer from menorrhagia can experience symptoms such as intense cramping, abdominal and deep pelvic pain, exhaustion, dyspnea (shortness of breath), fainting spells and angina or chest pain. In addition, the menstrual bleeding in menorrhagia can include clots or be thicker than normal blood, and may be so excessive that women suffer from anemia.

Women diagnosed with menorrhagia or AUB have limited treatment options available to them. Treatment options typically follow a progression that begins with drug therapy and ends with invasive surgery. The first line of treatment for excessive or abnormal uterine bleeding is the use of pharmaceutical or medical therapy. A variety of drugs can be used to help control the condition, including hormonal, non-steroidal anti-inflammatory (NSAID), low dose oral contraceptives, and antifibrinolytic drugs, all of which require a continuous regimen. One of the most common drug regimens is prescription low-dose oral contraceptive pills, which use estrogen to prevent ovulation, and thus reduce menstrual bleeding. However, there is not much clinical data backing the effectiveness of this therapy. Another commonly prescribed therapy for menorrhagia is a progesterone, or progestin regimen. Progestins must typically be taken in high doses to relieve menorrhagia symptoms, though even at higher dosage levels, these agents have not been proven more effective than an NSAID (aspirin, acetaminophen). Both oral contraceptives and progestins have been shown to produce adverse side effects such as weight gain, moodiness, nausea, headaches, and bloating. In addition, to OC's and progestin, physicians also prescribe gonadotropin releasing hormone (GnRH) agonists, such as Lupron, for treatment of AUB. GnRH agonists inhibit the release of the follicle stimulating (FSH) and luteinizing hormone (LH) that are produced by the pituitary gland and stimulate estrogen production in the ovaries. Cutting off the production of estrogen creates a menopausal effect in women, and therefore significantly reduces the volume of blood loss during menses. While GnRH agonists represent an effective therapy, these drugs can produce severe side effects in women, including bone density loss, mood swings and menopausal symptoms such as hot flashes. As a result of these side effects, GnRH agonists can only be prescribed for a short term (3-6 month) usage. While drug therapy may be a good option for women that are of fertile age and who wish to have a family, the combination of negative side effects, poor patient compliance, and the fact that drug therapy is only effective for approximately 50% of women has created a need for better options in treating AUB.

Dilation and Curettage (D&C) is another treatment option. The D&C, which is most commonly performed by gynecologists for diagnostic purposes, begins with the physician using a speculum to fully dilate the cervix. Once the cervix is dilated, the physician passes a curette into the uterus to perform mechanical scraping of the endometrium away from the uterine walls. While this procedure can improve the symptoms of abnormal bleeding, it usually only provides a temporary solution which remains effective for a few menstrual periods. Therefore, due to its short-term effectiveness, the D&C is not typically viewed as an effective therapy for abnormal uterine bleeding.

Surgery has become the primary treatment for AUB when a patient does not respond to or cannot tolerate conventional medical therapy. A hysterectomy, or complete removal of the uterus, is currently the most common surgical therapy for women who no longer wish to have children and experience excessive menstrual bleeding. There are several versions of hysterectomy surgery, including abdominal, vaginal, and laparoscopically assisted vaginal procedures. Complications associated with this procedure include infection, excessive bleeding, deep vein thrombosis, pulmonary embolism, urinary retention, pelvic adhesions and damage to adjacent organs such as the bladder or bowel. A hysterectomy is performed under general anesthesia and typically requires several days of hospital recovery and 6 to 10 weeks of home recuperation. In addition to the typical morbidity that is commonly associated with any major surgery, hysterectomy patients have reported a number of long-term physical and psychological side effects, including conditions such as depression and sexual dysfunction. In other cases, women who have hysterectomies experience menopause like symptoms arising from hormonal imbalances, even if surgery does not involve removing the ovaries. This is due to the fact that the blood supply to the ovaries changes with removal of the uterus. These women are typically prescribed to a hormone replacement regimen to offset hot flashes, declining bone density, headaches and moodiness that are commonly associated with menopause. Additionally, the hysterectomy can also lead to other conditions such as pelvic floor disorders and urinary incontinence. While hysterectomy is absolutely the appropriate therapy for women with any kind of uterine or ovarian cancer, patients and physicians alike are beginning to question the appropriateness of removing 100% of the uterus in order to treat menorrhagia that arises from only 5% of the organ.

Hysteroscopic endometrial ablation is a less invasive alternative to hysterectomy that utilizes a fiber optic telescope that is used to visualize the uterine cavity and a resectoscope and electro-cautery tools are used to ablate or destroy the functional layer of the endometrium, thus preventing abnormal uterine bleeding. Hysteroscopic endometrial ablation, typically performed on an outpatient basis, produces far less discomfort and requires a significantly shorter recovery period than a hysterectomy. This procedure is also considered safer than a hysterectomy, and it keeps both the uterus and the hormone levels in tact. However, because the procedure destroys the lining of the uterus, it is indicated for women who no longer wish to have children.

Hysteroscopic endometrial ablation is commonly performed using two techniques, roller ball endometrial ablation (REA) and transcervical resection of the endometrium (TCRE). An REA procedure utilizes a rolling electrode mounted to a hysteroscope to deliver an electrical current to the endometrial tissue, while TCRE employs a wire resection loop that when electrically activated, scrapes away sections of the endometrium. While REA and TCRE can be performed separately, they are often used concomitantly to obtain the best results. In many cases, the surgeon will first use the roller ball to ablate tissue in the areas of the uterus that are difficult to reach, then will use the loop, ablating endometrial tissue in rows across the uterus. Performed on an outpatient basis, the typical endometrial ablation procedure tends to last between 30 and 60 minutes and almost always involves the use of general anesthesia. Patients remain in the outpatient setting for a few hours post-op and can resume normal activity within two to three days following surgery. The procedure produces amenorrhea, or complete cessation of blood flow, in approximately 30% to 50% of the cases. The success rate for hysteroscopic endometrial ablation are lower than those produced by hysterectomy, which is 100% successful at creating amenorrhea. Approximately 15% of patients receiving hysteroscopic endometrial ablation will require a repeat ablation procedure at some point in the future, with repeat procedures more common among younger women. Another drawback of hysteroscopic endometrial ablation is that performing the procedure requires extensive skill and experience with the operative hysteroscope. It is estimated that less than 20% of practicing gynecologists have the necessary hysteroscopic skills to perform an endometrial ablation.

Realizing that the benefits of hysteroscopic endometrial ablation were limited by a challenging operative procedure, there have been several less invasive and less skill-dependent technologies and procedures developed. Known as global endometrial ablation, these techniques ablate endometrium tissue in a simple and uniform manner and produce clinical efficacy that is similar to hysteroscopic endometrial ablation. Global endometrial ablation involves transcervical placement of a thermal probe or balloon into the uterine cavity typically without the use of a hysteroscope. Once in position, the device delivers thermal energy in any one of the forms of radio frequency, hot saline, microwave, and cryogenic, etc. to the endometrium, resulting in tissue ablation. These procedures avoid the use of fluid distention used during hysteroscopic endometrial ablation, which eliminates the life threatening condition of hyponatremia and hypervolemia and importantly these procedures do not require operative hysteroscopic skill, opening up the treatment to virtually all gynecologists.

Because global endometrial ablation has a similar clinical efficacy to hysteroscopic endometrial ablation, it also results in relatively low amenorrhea rates ranging from 13% to 40%. Additionally, it is estimated that approximately 20% of global endometrial ablation patients will ultimately require a hysterectomy to put an end to their abnormal uterine bleeding.

Various methods have been proposed, each utilizing different types of energy sources to reduce the symptoms associated with AUD. However, each of these methods has met with various success rates at attaining amenorrhea. For example, J & J Gyncare uses a heated balloon that results in a 13% amenorrhea rate. Higher amenorrhea rates are achieved with a Boston Scientific device that utilizes a hot saline energy source (40% amenorrhea rate); and a Cytyc device that utilizes a RF mesh energy source (40% amenorrhea rate). Notably none of these devices achieves an amenorrhea rate greater than 40%. Additionally, these low amenorrhea rates ultimately cause up to 20% of patients to have a hysterectomy.

In view of the above limited success rates, there is a need for a minimally invasive device and method to treat normal abnormal intrauterine bleeding with a device that possesses a high success rate at treating amenorrhea and has minimal side-effects or related complications.

### SUMMARY

The methods and devices described below provide for near 99% amenorrhea rates in women. The device comprises a porous biomaterial implant that is capable of being positioned into the ablated uterine cavity of a female patient. The porous biomaterial implant can be variously shaped. Once positioned in the uterine cavity, the porous biomaterial implant expands or is inflated within the uterine cavity. The ablated uterine tissue then grows within the porous implant.

The uterine cavity is prepared by performing endometrial ablation to at least the inferior or lower portion of the uterine cavity. A porous biomaterial implant that is variously shaped is then placed within the uterine cavity. Placement of the porous biomaterial causes the uterine cavity walls to coapt, achieving complete occlusion of the uterine cavity and prevention of endometrial regrowth. This results in amenorrhea, resulting in a reduced number of hysterectomies in patients. Alternatively, this can result in an effective contraceptive method.

### BRIEF DESCRIPTION OF THE DRAWINGS

FIG. 1 is a partial view of the female reproductive system;

FIG. 2a is a front view of a triangular configuration of a porous biomaterial implant;

FIG. 2b is a cut away view of the implant of FIG. 2a;

FIG. 3a is a front view of an implant containing a connection element;

FIG. 3b is a cut away view of the implant of FIG. 3a;

FIG. 4a is a front view of an implant with walls of varied thickness to control areas of expansion;

FIG. 4b is a cut away view of FIG. 4b;

FIG. 5 is a partial view of an implant positioned within the uterine cavity of a female;

FIG. 6a is a front view of a triangular shaped configuration of a porous implant that contains a T-shaped internal frame;

FIG. 6b is a cut away view of FIG. 6a;

FIG. 7a is a front view of a triangular shaped configuration of a porous implant that contains a V-shaped internal frame;

FIG. 7b is a cut away view of FIG. 7a;

FIG. 8 a is a front view of a triangular shaped configuration of a porous implant that contains a triangular shaped internal frame;

FIG. 8 b is a cut away view of FIG. 8 a; and

FIGS. 9a through 9e illustrate a porous implant capable of being inserted transcervically via a cannula and applicator unit into a pretreated uterus.

### DETAILED DESCRIPTION OF THE INVENTIONS

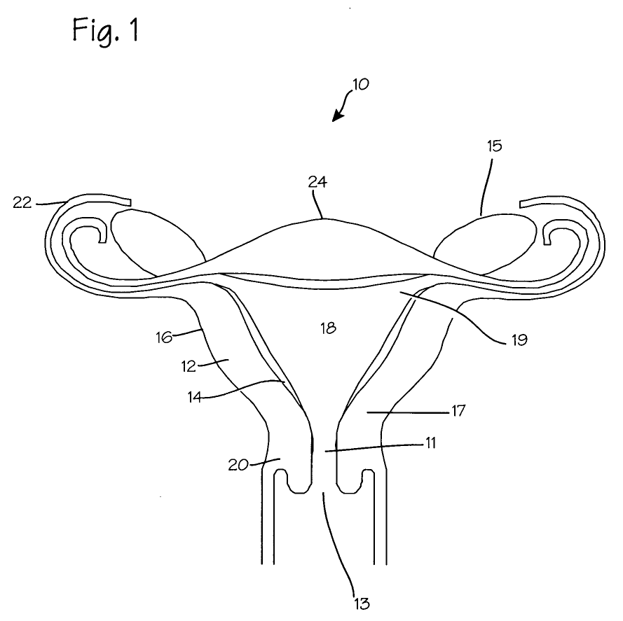

FIG. 1 is a partial view of the female reproductive system. This figure illustrates the environment for which the devices and methods have been developed. The uterus, which is also referred to as the womb, 10 is the pear shaped hollow, muscular organ. It consists of three primary layers, the peritoneum, which is the outermost layer of the organ, the myometrium, the muscular middle layer of the organ, and the endometrium, which is highly vascular mucous membrane that serves as the interior lining of the uterus. The thick mucosal coat, the myometrium 12, is a cavity having an inner mucosal lining of variable thickness called the endometrium 14, a thin membrane that lines the abdominal and pelvic cavities called the peritoneum 16, and a cavity referred to as the uterine cavity 18. The endometrium is the layer that supports and provides nourishment to a developing embryo during pregnancy, and is also the layer that sheds away during menstruation. The endometrium, which is approximately 4 to 5 millimeters thick and accounts for approximately 5% of the total uterus, consists of two discrete layers, the functional and basal layers, that are highly responsive to hormonal activity. The basal layer borders the myometrium and serves as a foundation for the functional layer, which, if a woman is not pregnant, sloughs off during menstruation and is regenerated with each 28-day menstrual cycle. The cervix 20 defines the cervical canal which is an opening to the vagina. The part of the cervical opening located at the bottom apex of the uterine cavity is the internal cervical ostium 11 and the part of the cervical opening located in the vagina is the external cervical ostium 13. The ovary 15 is the organ that produces one or more eggs during every woman's reproductive life. The uterine tube 22 is either of a pair of tubes conducing an egg produced during a woman's reproductive cycle from the ovary to the uterus. The fundus 24 is the upper, rounded portion of the uterus. The lower or inferior portion of the uterine cavity is commonly called the uterine isthmic segment 17. The top or superior two corners of the uterine cavity where the uterine tubes enter the cavity are called the cornual regions 19.

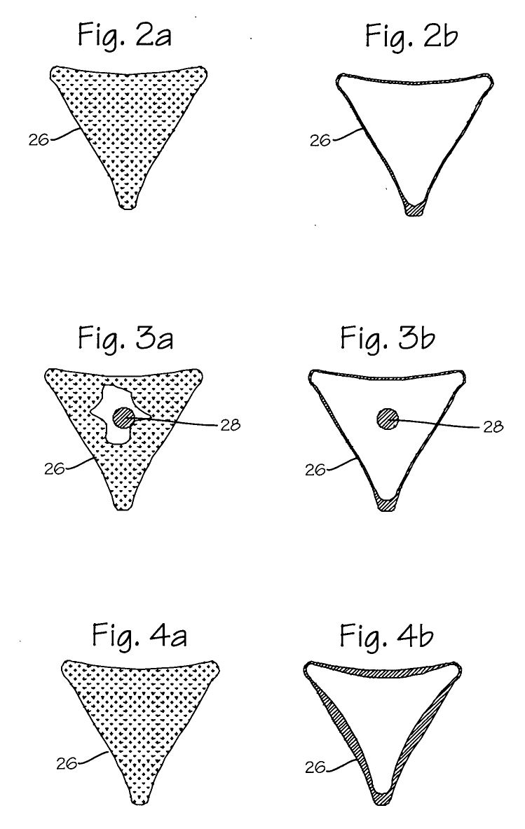

FIGS. 2a through 4b illustrate various configurations of the porous biomaterial implant. The device of the present invention comprises a porous biomaterial implant that is capable of being positioned into the pretreated uterine cavity 18 of a female patient. The implant can be variously shaped, and may be conformal, non-conformal, or semi-conformal when in an open position within the uterine cavity. The implant is pliable and can be folded, rolled, or otherwise manipulated. Once positioned in the uterine cavity, the porous biomaterial implant expands into its preformed shape within the uterine cavity. The pretreated uterine tissue then grows within the porous implant. Subsequent healing includes ingrowth of vascularized structures into the connective tissue residing in the pores of the implant.

FIG. 2a is a front view of a triangular configuration of a porous biomaterial implant and FIG. 2b is a cut away view of FIG. 2a. The implant 26 may be comprised of a porous bladder or balloon like structure. The outer membrane of the balloon implant may be comprised of porous material while the internal membrane is solid in order to maintain pressure and assist with forming the implant to the internal dimensions of the uterine cavity. The configuration of this implant 26 is of a generally triangular shape because this general shape fits bests into the uterine cavity. The bottom apex of the triangular implant is positioned at the internal cervical ostium of the female patient and the top corners of the triangle are positioned into the cornual regions. The implant 26 is manufactured of a biomaterial from any of several known materials used for medical devices. By way of example, and not as an exhaustive list, the following materials are suitable for use in manufacture of the implant: plastics such as polyethylene, polycarbonate, polypropylene, ionomer, polyester, polyethylene terepathalate, polybutyleneterepathalate, polyurethane, epoxy, silicone, polytetrafluoroethylene, latex, natural rubbers, polyvinyl alcohol, polyvinyl acetate, thermoplastic elastomers and fluoropolymers. The implant can also be made to be fluoroscopically and/or ultrasonically visible by the compounding or loading of agents within the biomaterial. Examples of agents are barium sulfate, bismuth subcarbonate, platinum and gold powder, micro glass beads, etc. The implant can also be loaded or coated with medications and agents that improve ingrowth rates and quality and decrease the chances of infections such as iodine and vascular endothelial growth factor (VEGF). Additionally, many different metals and ceramics are suitable for manufacture of the implant material.

Alternatively, the implant may be manufactured from one of many different porous biomaterials with a pore size, architecture and chemistry that facilitate cellular ingrowth into the material. Some examples include sintered plastics such as polyethylene, polypropylene, polytetrafluoroethylene. Another example of a porous material is expanded polytetrafluoroethylene which is made from a stretching or expanding technique. Yet another example of a porous material is silicone or other similar material manufactured into a finished porous form using any of the known techniques leaching out crystals to create the porosity of the material. Types of crystals that may be used are salt or sugar crystals. Alternatively, the material may be fibrous, such as Dacron fibers (PET). The pores on the biomaterial may exist on the exterior surface or interior surface of the implant. The pores may be interconnected to allow communication between each other. Pore sizes typically range from 1 micron to approximately 400 microns. The implant may also be variously shaped such as uterine shaped. In such a configuration, the overall shape of the implant is that of a female uterus, however, the implant is still generally triangular in shape such that it possesses a bottom apex and two top corners. Alternatively, the implant can be of a mushroom shaped configuration. Once again this shape is easily insertable into a female uterus with the bottom apex insertable through the cervix of the female patient and the top sides of the mushroom positioned into the corneal regions.

FIG. 3a is a front view of a connection or bridge 28 placed between an inflatable balloon configuration porous implant 26. The outside surface of the implant is comprised of a non-conformable porous bladder or balloon like structure. FIG. 3b is a cut away view of the implant of FIG. 3a. A connection or bridge is placed between the balloon walls in order to prevent the mid section of the balloon from over expansion when in the uterine cavity. In this illustration there is only one bridge or connection utilized, but more than one may be used in order to control the expansion of the implant.

FIG. 4a is a front view of an implant 26 with walls of varied thickness to control areas of expansion. FIG. 4b is a cut away view of the implant of FIG. 4a. The implant is comprised of walls of varying thickness at different points of the implant. The variations in thickness are used to control different areas of expansion of the implant. The areas of thicker walls expand less that the areas of thinner walls.

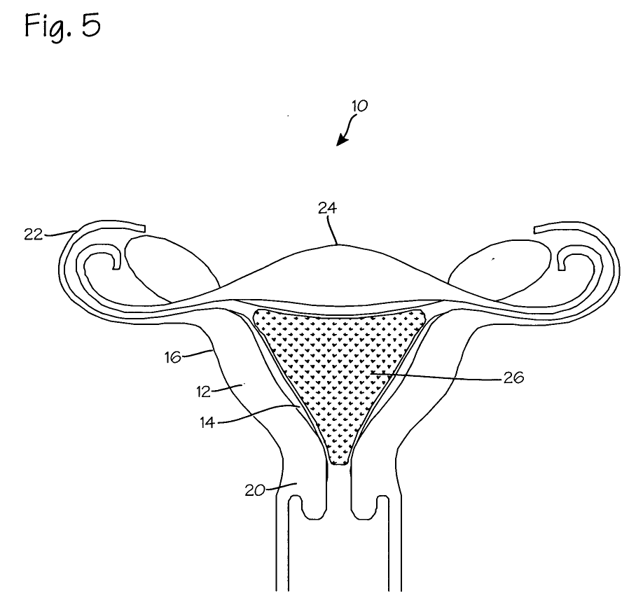

FIG. 5 illustrates what the implant looks like once properly positioned within the uterine cavity and inflated. The implant is filled to expand the balloon and fill the uterine cavity.

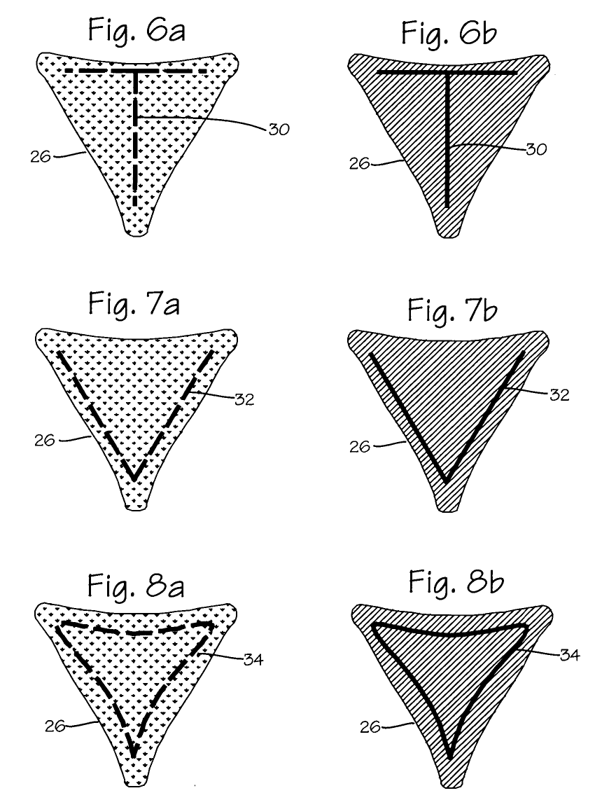

The implant 26 may also contain a frame or internal support structure that aids in correctly positioning the implant into the uterine cavity. FIGS. 6a through 8 b illustrate implants 26 with different internal frame designs. FIG. 6a is a front view of a triangular shaped configuration of a porous implant that contains a T-shaped internal frame 30. FIG. 6b is a cut away view of the implant of FIG. 6a. FIG. 7a is a front view of a triangular shaped configuration of a porous implant that contains a V-shaped internal frame 32. FIG. 7b is a cut away view of the implant of FIG. 7a. FIG. 8 a is a front view of a triangular shaped configuration of a porous implant that contains a triangular shaped internal frame 34. FIG. 8 b is a cut away view of FIG. 8 a. While the internal frames may be configured of various shapes, they are all bendable and resilient to allow the frame to be manipulated. This allows the implant to be folded during insertion of the implant into the uterine cavity. Once the implant is positioned within the uterine cavity, the internal frame support structure assists in maintaining the proper position of the implant within the uterine cavity.

The support frame design is constructed to assist with the proper deployment and placement of the implant within the uterine cavity. The frame should be bendable, resilient, and capable of being manipulated. However, the support frame should not be too stiff or sharp such that it causes any end or edge to extrude from the implant through the porous biomaterial surface. Examples of the frame material include semi rigid and resilient plastic such as polyethylene, polypropylene, fluoropolymers, polyurethanes, polyethylene terepathalate, nylon, polybutyleneterapathalate, and ionomers. Additionally, the frame can be constructed from round or flat metal wire such as nickel titanium, Nitinol®, MP35N, Elgiloy, stainless steel, and piano wire.

The method of use requires pretreatment of the uterine cavity prior to placement of the implant. Prior to insertion of the implant, removal or destruction of at least the isthmic or lower uterine cavity must be accomplished. This is performed by any of several methods of endometrial ablation. The pretreatment of the uterine cavity prior to implantation assists with the ultimate incorporation of the implant into the uterine myometrium. The removal or destruction of the endometrium with an acute inflammation response allows the myometrium to grow within the porous implant once properly positioned. The pretreatment also assists in preventing the endometrium from regenerating and thus result in amenorrhea in the patient. Complete treatment of the entire uterine cavity is not required. Only complete treatment of the lower apex or isthmic region of the uterine cavity is required. This causes the corneal areas and the superior areas of the endometrium tissue that have not been treated to become non-functional. Therefore, incomplete treatment of the superior areas of the uterine cavity with sufficient treatment of the isthmic or lower cavity will still result in amenorrhea.

The pretreatment is preferably conducted by endometrial ablation of the uterine cavity. This can be accomplished with the use of lasers, resection loops, roller ball electrodes or the like to destroy the endometrium. This can be accomplished with or without the use of a hysteroscope. Pretreatment can also be conducted using any of the commercially available global endometrial devises. Alternatively, the pretreatment of the uterine cavity may be accomplished by medical therapy treatment involving use of non-steroidal anti inflammatories low dose oral contraceptives or gonadotropin releasing hormone agonists, such as Lupron. Also, the pretreatment can further be accomplished by Dilation and Curettage prior to placement of the implant. Additionally, pretreatment can be accomplished by delivery of a caustic agent such as ethanol or tetracycline. Also, pretreatment may be accomplished by use of the balloon implant itself. Hot water or saline may be circulated through an implant placed within the uterine cavity. The temperature of the hot liquid within the implant results in ablation of the endometrial tissue. Alternatively, RF wires, bands or mesh may be placed on the outside surface of the implant. Once the implant is properly positioned and inflated within the uterine cavity, RF energy can be delivered through electrodes to the endometrial tissue. Once the endometrium is appropriately ablated, the electrode containing surface of the implant may be removed from the patient. Alternatively, a laser can be delivered through the implant to ablate the tissue. Small micro reflectors can be positioned throughout the walls of the implant. As the laser light hits the reflectors, the laser becomes redirected towards the endometrium and adequately pre-treats the area. Alternatively, by inflating the implant to a higher pressure than the cavity is accustomed, necrosis of the endometrium will naturally occur as a result of phenomenon called pressure necrosis. This results in the death of the endometrium and causes the underlying tissues layer to grow into the pores of the implant. Another added benefit of this particular method is that it does not require the delivery of a thermal energy or accessory equipment such as an energy generator.

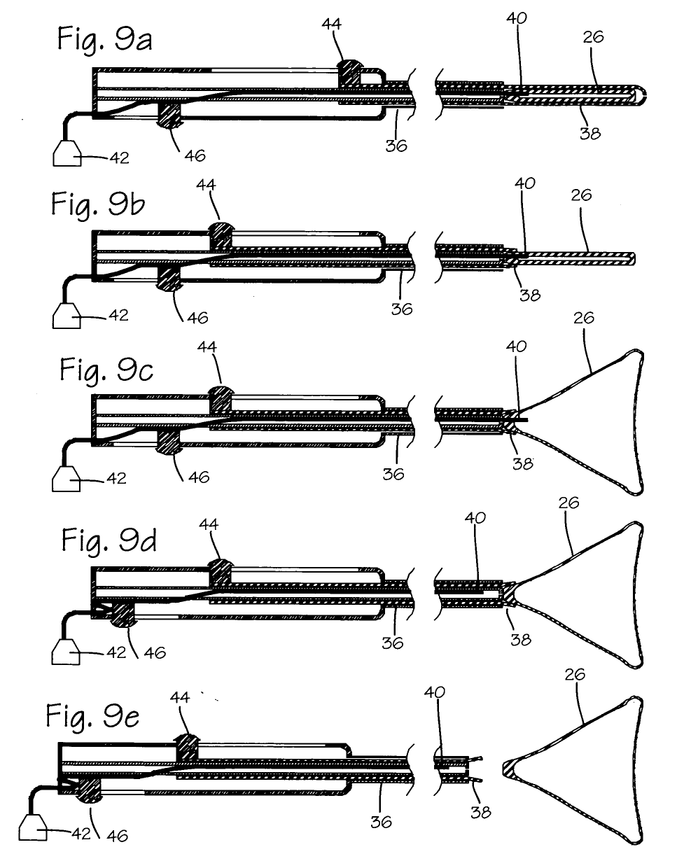

Once the endometrial ablation is performed, the implant is ready to be inserted into the uterine cavity. FIGS. 9a through 9e illustrate a device used in the method of inserting the implant into the uterine cavity with the assistance of a cannula. FIG. 9a illustrates a cannula 36 having a first diameter. The cannula can have a limiter or stopper placed on the outside diameter (not shown) that prevents the device from being inserted too deep into the cavity. Limiter has a diameter that is bigger than the external cervical ostium, that once the device is in proper position, the limiter prevents the device from being positioned deeper in the cavity. The cannula contains retractable applicator unit 38 having a first diameter, the first diameter of the applicator unit being less than the diameter of the cannula. The applicator unit extends beyond the end of the cannula and projects a distance from the end of the cannula. Contained with the applicator unit is a compressed implant. The compressed implant is releasably connected at one end to an inflation means 40, which is operably connected to a luer fitting 42. The retractable applicator unit is attached to a first stopper 44 that assists in restricting the movement of the applicator unit to within a particular range so that the applicator unit is not advanced too far into the patient. Additionally, the inflation means is also attached to a second stopper 46 that assists in retracting the inflation means once the implant has been adequately inflated.

The cannula and applicator unit are transcervically inserted into the pretreated uterine cavity of a patient. FIG. 9a illustrates an implant 26 that is contained within the applicator unit. The implant is manipulated to fit within the applicator unit by being preloaded within the applicator unit to assist with crossing the cervix. The compressed implant is releasably engaged at one end to an inflation means 40. FIG. 9b illustrates the applicator unit being retracted from the uterine cavity and back into the cannula to expose the compressed implant. The applicator unit is connected to a first stopper that assists in restricting the movement of the applicator unit within a particular range. The applicator movement retracts up until a point when the stopper prevents further retraction. At this point, the compressed implant is entirely exposed within the uterine cavity. FIG. 9c illustrates the complete retraction of the applicator unit and the inflation of the implant. The implant may then be inflated with air, other gases, water, saline, mineral oil, silicone oil, silicone plastic, foaming plastic or the like in order to inflate the balloon implant into an expanded position. The inflated implant may be permanently or temporarily secured shut so that the material does not escape. FIG. 9d illustrates an implant that is permanently secured so that deflation of the implant does not occur. However, the physician may also temporarily secure the implant and the patient may return at a later date so that the treating physician can remove the filler material. Alternatively the filler material may be combined with anti-infection agents and allowed to slowly release from the implant. The filler material would slowly leak out of the cervix end of the balloon implant, pass through the cervix and into the vagina to be discharged. The rate of release can be varied. This allows the balloon implant to slowly deflate, eliminating pressure on the uterine cavity and reducing the possibility of uterine cramps in the patient. Alternatively, a second implant can be placed within another implant (not shown). The interior balloon implant may be inflated such that it causes the exterior balloon to expand within the uterine cavity. The patient may return after a period of time and have the interior balloon removed but maintain the exterior balloon. An added benefit of this method is that pretreatment of the endometrium may not be required due to the phenomenon of pressure necrosis of the endometrium that results in the necrosis of the endometrium and healthy ingrowth of the underlying tissue. Alternatively, where an implant with an internal support frame is used, inflation of the implant is not required. The compressed implant is exposed within the uterine cavity and fully expands into its completely retracted position upon release from the applicator unit.

FIG. 9e illustrates the implant being released from the cannula and applicator assembly. At this point the implant remains embedded within the uterine cavity. This allows the tissue to properly grow into the porous surface over a short period. The introduction of the implant into the pretreated uterine cavity results in epitaxial expansion of the implant into the uterus. Over time, the pretreated tissue grows into the porous surface of the implant, preventing endometrial tissue from regenerating, resulting in amenorrhea of the patient.

Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A method of creating amenorrhea in a female patient comprising the steps of:
pre-treating at least the inferior uterine cavity of the patient wherein pre-treating comprises destroying the endometrial tissue of the uterine cavity;
introducing a porous implant into the pretreated uterine cavity wherein the implant forms epitaxial expansion into the uterine cavity; and
allowing the uterine cavity walls to coapt into the implant, achieving complete occlusion of the uterine cavity and prevention of endometrial growth.

2. The method of claim 1 further comprising the step of inflating the implant after it has been introduced into the pretreated uterine cavity.

3. The method of claim 2 further comprising the step of releasably sealing the implant after introduction of the implant into the pretreated uterine cavity.

4. The method of claim 1 wherein the step of pre-treating the uterine cavity is achieved by endometrial ablation.

5. The method of claim 1 wherein the step of pre-treating the uterine cavity is achieved by the application of a non-steroidal anti-inflammatory.

6. The method of claim 1 wherein the step of pre-treating the uterine cavity is achieved by the application of an oral contraceptive.

7. The method of claim 1 wherein the step of pre-treating the uterine cavity is achieved by dilation and curettage.

8. The method of claim 1 wherein the implant has shape that approximates a triangle.

9. The method of claim 1 wherein the implant comprises a biomaterial.

10. The method of claim 1 wherein the implant comprises a bladder.

11. The method of claim 1 wherein the implant further comprises an internal support structure contained within the porous implant.

12. A method of creating amenorrhea in a female patient comprising the steps of:
pre-treating at least the inferior uterine cavity of the patient via endometrial ablation;
introducing a porous implant into the pretreated uterine cavity wherein the implant forms epitaxial expansion into the uterine cavity;
inflating the implant after it has been introduced into the pretreated uterine cavity and releasably securing the inflated implant; and
allowing the uterine cavity walls to coapt into the implant, achieving complete occlusion of the uterine cavity and prevention of endometrial growth.

13. The method of claim 12 wherein the implant has shape that approximates a triangle.

14. The method of claim 12 wherein the implant comprises a biomaterial.

15. The method of claim 12 wherein the implant comprises a bladder.

16. The method of claim 12 wherein the implant further comprises an internal support structure contained within the porous implant.

17. A method of creating amenorrhea in a female patient comprising the steps of:
introducing a porous implant into the uterine cavity of the female patient;
causing the implant to destroy at least the inferior uterine cavity and the endometrial tissue contained therein;
allowing the uterine cavity walls to coapt to the implant, achieving complete occlusion of the uterine cavity and prevention of endometrial growth.

18. The method of claim 17 wherein the step of destroying the uterine cavity is performed by endometrial ablation.

19. The method of claim 17 wherein the implant comprises an expandable bladder.

20. The method of claim 17 further comprising the step of inflating the expandable bladder after destruction of the endometrial tissue.

21. The method of claim 20 wherein the implant contains at least one connection between balloon walls to minimize expansion of the bladder walls.

22. The method of claim 20 wherein the implant walls comprise varied thicknesses to minimize expansion of the bladder walls.

23. The method of claim 19 wherein the step of causing the implant to destroy at least the inferior uterine cavity and the endometrial tissue contained therein is performed by filling the bladder with a gas or liquid sufficiently hot to destroy the endometrial tissue.

24. The method of claim 17 wherein the step of causing the implant to destroy at least the inferior uterine cavity and the endometrial tissue contained therein is performed by means capable of conducting electrical current positioned on the outside surface of the bladder.

25. The method of claim 20 wherein the implant further comprises a second bladder contained within the first bladder.

26. The method of claim 25 wherein the step of causing the implant to destroy at least the inferior uterine cavity and the endometrial tissue contained therein is performed by filling the second bladder with a gas or liquid sufficiently hot to destroy the endometrial tissue.

27. The method of claim 20 wherein the expandable bladder is permanently sealed upon introduction of the gas.

28. The method of claim 20 wherein the expandable bladder is releasably sealed upon introduction of the gas.
